(19) United States
(12) Patent Application Publication
(10) Pub. No.: US 2015/0297296 A1
(43) Pub. Date: Oct. 22, 2015

(54) PACKAGING ASSEMBLY FOR DRUG DELIVERY DEVICES

(71) Applicant: Sanofi-Aventis Deutschland GmbH, Frankfurt (DE)

(72) Inventors: Udo Stauder, Frankfurt am Main (DE); Ivana Berger, Frankfurt am Main (DE); Dagmar Weber, Frankfurt am Main (DE)

(21) Appl. No.: 14/646,478

(22) PCT Filed: Dec. 19, 2013
(86) PCT No.: PCT/EP2013/077308

§ 371 Date: May 21, 2015

(30) Foreign Application Priority Data
Dec. 20, 2012 (EP) 12198365.4
Mar. 11, 2013 (EP) 13158638.0

(51) Int. Cl. A61B 19/02; B65D 77/04; B65D 25/20; A61J 1/05

(52) U.S. Cl. CPC A61B 19/026; A61J 1/05; B65D 77/04; B65D 25/205; A61B 2019/0202; A61B 2019/0278; A61J 2205/30

(57) ABSTRACT

A packaging assembly to accommodate at least two drug delivery devices is presented having an inner packaging to accommodate a first drug delivery device, and an outer packaging to accommodate a second drug delivery device and to accommodate the inner packaging.

### CROSS REFERENCE TO RELATED APPLICATIONS

The present application is a U.S. National Phase Application pursuant to 35 U.S.C. §371 of International Application No. PCT/EP2013/077308 filed Dec. 19, 2013, which claims priority to European Patent Application No. 12198365.4 filed Dec. 20, 2012 and European Patent Application No. 13158638.0, filed Mar. 11, 2013. The entire disclosure contents of these applications are herewith incorporated by reference into the present application.

### TECHNICAL FIELD

The present invention relates to the field of packaging assemblies and in particular to packaging assemblies adapted to accommodate at least two drug delivery devices, such like injection devices.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, in particular pen-type injectors have to meet a number of user-specific requirements. For instance, with patient's suffering chronic diseases, such like diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Moreover, a dose setting as well as a dose dispensing procedure must be easy to operate and has to be unambiguous.

Typically, such devices comprise a housing or a particular cartridge holder, which is adapted to receive a cartridge at least partially filled with the medicament to be dispensed. The device further comprises a drive mechanism, usually having a displaceable piston rod which is adapted to operably engage with a piston of the cartridge. By means of the drive mechanism and its piston rod, the piston of the cartridge is displaceable in a distal or dispensing direction and may therefore expel a predefined amount of the medicament via a piercing assembly, which is to be releasably coupled with a distal end section of the housing of the drug delivery device.

The medicament to be dispensed by the drug delivery device is provided and contained in a multi-dose cartridge. Such cartridges typically comprise a vitreous barrel sealed in distal direction by means of a pierceable seal and being further sealed in proximal direction by the piston. With reusable drug delivery devices an empty cartridge is replaceable by a new one. In contrast to that, drug delivery devices of disposable type are to be entirely discarded when the medicament in the cartridge has been completely dispensed or used-up.

Drug delivery devices may also comprise conventional syringes, in particular pre-filled syringes that are intended for single use. Moreover, injection-based treatment of patients may require to make use of different pharmaceutical substances or medicaments to be injected or dispensed by different injection devices or drug delivery devices, respectively.

Typically, at the beginning of a therapy a first medicament may have to be administered before a second medicament should be used later on. In many cases, the two medicaments to be administered sequentially are commercially distributed as a treatment kit, in which first and second medicaments to be delivered by respective first and second drug delivery devices mutually complement. Since the two medicaments together with respective drug delivery devices are mutually correlated it is only natural, that said kit consisting of at least two medicaments and drug delivery devices is commercially distributed in form of a common packaging assembly.

Providing at least two different drug delivery devices in a common packaging assembly may be confusing to the end user or patient. In particular with self-medication or home treatment the patient himself has to correctly identify the particular drug delivery device which is to be used initially. Even though the packaging assembly may be provided with respective instructions about general usage of first and second drug delivery devices, the patient himself, for whichever reason, may disregard or misunderstand respective instructions. Hence, there is a certain risk, that the two drug delivery devices are mixed up and that they are used in an inappropriate way.

It is therefore an object of the present invention to improve patient safety and to reduce a risk of health for medical treatment making use of multiple medicaments and respective drug delivery devices. It is a particular aim of the present invention to provide an intuitive and unequivocal approach to instruct a user in regard of the order of usage of at least two drug delivery devices provided in a common packaging assembly. Respective instructive means should be easy to implement in a cost efficient way.

### SUMMARY

In a first aspect, the invention relates to a packaging assembly to accommodate at least two drug delivery devices. The packaging assembly comprises an inner packaging or package to accommodate a first drug delivery device and further comprises an outer packaging which is adapted to accommodate a second drug delivery device and to accommodate further the inner packaging, e.g. containing the first drug delivery device.

In this way, the packaging assembly features a nested arrangement of an inner and an outer packaging. When opening the outer packaging, only the second drug delivery device is directly accessible to be used while the first drug delivery device remains packed and sealed by the inner packaging contained in the outer packaging. Such an interleaved configuration of inner and outer packaging provides an intuitive approach to indicate to a user, that the second drug delivery device should be used prior to the first drug delivery device, which is separately packed by the inner packaging.

When opening the outer packaging there is only one, namely the second drug delivery device of a plurality of drug delivery devices which is ready to use. The user may then intuitively take the second drug delivery device. Hence, a risk of interchanging first and second drug delivery devices can be reduced, simply by wrapping the first drug delivery device in a separate inner packaging.

In a further embodiment, the inner packaging is adapted to completely enclose the first drug delivery device. Additionally, the outer packaging is adapted to accommodate and to receive the second drug delivery device in a configuration, in which the second drug delivery device is ready to use. When assembled in the outer packaging, the second drug delivery device can be free of any further packaging means. The second drug delivery device can be arranged outside the inner packaging but inside the outer packaging.

In the present context completely enclosing the first drug delivery device means that the device is completely surrounded by the inner packaging which may be open towards the top. In order to prevent inadvertent or non-intended removal of the first drug delivery device at least a section of the inner packaging's interior can be covered by a lid or by a fastening member. Hence, the inner packaging may comprise at least a circumferential side wall structure and a bottom portion. It may be only optionally provided with a lid.

In a further embodiment, the outer packaging comprises at least one fastening member to fix the second drug delivery device therein. Said fastening member can be adapted to releasably engage the second drug delivery device. If for instance the second drug delivery device is intended for multiple use, e.g. over a period of several days or weeks, the second drug delivery device can be repeatedly fastened and fixed inside the outer packaging in order to store first and second drug delivery devices in a well-defined order, e.g. in a refrigerated environment.

In a further embodiment, the outer packaging may also comprise at least one fastening member to fix the inner packaging therein. Here it is conceivable, that the inner packaging is removably attachable to the outer packaging. Alternatively, it is conceivable, that the inner packaging is non-reversibly attached and fixed to the outer packaging. Hence, when detaching the inner packaging from the outer packaging and, hence when a treatment on the basis of the first drug delivery device begins, the outer packaging may become superfluous, since a respective treatment on the basis of the second drug delivery device should have already terminated.

Additionally or alternatively it is also conceivable, that the inner packaging is non-releasably engaged with the outer packaging. Removal of the first drug delivery device from the packaging assembly may therefore come along with opening of the inner packaging in order to take out the first drug delivery device from the inner packaging. Here, a removal and separation of inner and outer packagings is not required in general.

According to another embodiment, the inner packaging comprises at least one fastening member to fix the first drug delivery device therein. In this way, the inner packaging may be separately opened, e.g. by lifting a respective lid to provide access to the interior of the inner packaging. When having at least one fastening member provided in the inner packaging, the first drug delivery device can be securely attached therein either in a reversible or non-reversible way.

In a further embodiment the inner packaging comprises a bottom portion as well as a first sidewall portion and a second sidewall portion. First and second sidewall portions extend along opposite sides of the bottom portion.

Furthermore, the inner packaging comprises at least one front face extending between an end of the first sidewall portion, the second sidewall portion and the bottom portion. Typically, the outer shape and contour of the inner packaging is confined by the first and second sidewall portions, the bottom portion and the at least one front face extending there between. Moreover, first and second sidewall portions may be integrally formed with the bottom portion.

The first and second sidewall portions may be folded or bended along folding axes extending between and separating the bottom portion from the first and second sidewall portions. Typically, these folding lines or folding axes extend substantially parallel to each other to form a cubic-shaped packaging. The at least one front face comprises a surface normal extending substantially parallel to these folding lines or folding axes.

The inner packaging further comprises at least one fixing flap portion extending as an extension of the bottom portion and extending beyond the front face of the packaging. The at least one fixing flap portion extends outwardly, hence in a direction opposite to the interior of the inner packaging. The at least one foldable fixing flap portion is adapted to engage with the outer packaging that is in turn adapted to receive the inner packaging. Mutual engagement of the inner packaging's fixing flap portion with the outer packaging is typically obtainable when the inner packaging is disposed inside the outer packaging.

The at least one foldable fixing flap portion is adapted to positively engage with an interior structure of the outer packaging so as to assemble and to fix the inner packaging to and into the outer packaging without the necessity of adhesively attaching the inner packaging to the outer packaging. By making use of the foldable fixing flap portion, a well-defined, irreversible and non-releasable interconnection of the inner packaging inside the outer packaging can be attained, in particular when the inner packaging is completely inserted into the surrounding outer packaging.

In this way, the packaging assembly is adapted and particularly suitable to provide a nested arrangement of inner packaging and outer packaging. By means of the at least one foldable flap portion, the inner packaging can be non-reversibly attached and fixed to the interior of the outer packaging. The mutual fixing of inner and outer packaging can be designed such that removal of the inner packaging from the outer packaging inevitably leads to an at least partial destruction of the outer packaging. In an alternative embodiment it is also conceivable, that the foldable flap portion allows for a non-destructive removal of the inner packaging from the outer packaging.

Typically, the at least one fixing flap portion is adapted to positively engage with inside facing sidewall portions, in particular with an inside facing portion of a front face of the outer packaging featuring a stepped or recessed section, by way of which the bottom portion's foldable flap portion can be fixed or clipped to the outer packaging in a hooking manner.

According to another embodiment, the inner packaging comprises a first and a second fixing flap portion extending from opposite ends of the bottom portion and/or extending from oppositely located front faces. Typically but not necessarily, the bottom portion, as well as first and second sidewall portions are of rectangular geometry. By providing first and second fixing flap portions at opposite sides of the bottom portion, the inner packaging may engage with both opposite ends or with both oppositely located front faces with corresponding end faces of the exterior or outer packaging.

It is generally to be mentioned here, that the front faces of the inner packaging denotes an arbitrary side face of the inner packaging that is adapted and designed to engage with a corresponding side face of the outer packaging.

In a typical embodiment, the foldable fixing flap portions are provided at a longitudinal end of respective bottom and/or sidewall portions. Hence, the front face, which may be formed or constituted by mutually overlapping flap portions of the sidewall portions is located at a longitudinal end of the elongated and substantially rectangular packaging.

According to another embodiment the at least one fixing flap portion is integrally formed or connected with the bottom portion. Hence, the at least one fixing flap portion may be designed as a longitudinal extension of the bottom portion. Typically, first and second but longitudinally oppositely located fixing flap portions are provided and integrally formed with opposite longitudinal ends of the bottom portion.

Even though the at least one fixing flap portion extends as an extension of the bottom portion it does not necessarily have to be integrally formed or connected with the bottom portion. Instead and according to another embodiment, the at least one fixing flap portion may also be integrally formed with at least one of first and second sidewall portions or with first and/or second flap portions of first and/or second sidewall portions.

Depending on the shape, size and geometry of the item or items to be deposited in the inner packaging and/or the outer packaging various flap portions may be additionally or alternatively provided also at a short end section of the bottom portion and/or of the first and/or second sidewall portions.

By providing both opposite ends of the bottom portion with first and second fixing flap portions, respectively, opposite ends of the bottom portion of the inner packaging may non-releasably or non-reversibly engage with the outer packaging as soon as the inner packaging is put or inserted into the outer packaging.

Typically, the longitudinal dimensions of the outer packaging are substantially identical or match with the longitudinal extension of the inner packaging so that the foldable flap portions of the packaging's bottom portion are folded upwardly by oppositely located front faces of the outer packaging. In particular, the longitudinal extension of the interior of the outer packaging typically matches with the exterior longitudinal dimensions of the inner packaging's bottom portion plus the thickness of the inner packaging's front faces and the thickness of the bottom portion's foldable flap portions folded upwardly to overlap with and to abut against the inner packaging's front face.

In an assembly configuration, the foldable fixing flap portions of the inner packaging are typically sandwiched between the inside facing portion of the outer packaging's front face and the exterior portion of the inner packaging's front face. In this way and when longitudinally squeezed between oppositely located inside facing portions of the outer packaging's front faces, the folded fixing flap portions of the inner packaging are effectively hindered to fold downwardly to disengage from the outer packaging.

Typically, and according to another embodiment the first sidewall portion comprises at least one foldable flap portion overlapping with a foldable flap portion of the second sidewall portion or directly overlapping with the second sidewall portion. In this way, the foldable flap portions that are typically unitary or integrally formed with said first and/or second sidewall portions respectively, are adapted to form or to contribute to the front face of the inner packaging when arranged in an at least partially overlapping configuration.

According to another embodiment, at least one of first and second sidewall portions comprises first and second flap portions extending from opposite ends of the respective sidewall portion. Depending on the overall extension and geometry of respective flap portions it may be sufficient, when only the first sidewall portion is provided with first and second flap portions so that first and second flap portions can be arranged in an overlapping and e.g. two times folded manner with the second sidewall portion. In this configuration, the first and second flap portions of the first sidewall portions may provide or constitute the front face of the inner packaging extending between longitudinal end sections of first and second sidewall portions.

In an alternative embodiment, first and second sidewall portions at least comprise one, either a first or a second flap portion, so that e.g. a first flap portion of the first sidewall portion forms a first front face and wherein a second flap portion of the second sidewall portion forms a diametrically oppositely located second front face.

In a further embodiment, both, first and second sidewall portions each comprise first and second oppositely located flap portions that mutually overlap to form or to contribute to respective first and second, oppositely located front faces of the packaging.

In general and if not otherwise specified, overlapping portions of the inner packaging, and in particular mutually overlapping flap portions are typically interconnected and fixed to each other, e.g. by means of a glue or an adhesive. Moreover, the entire inner and/or outer packaging may be formed and provided by a single blank of a sheet material, such like cardboard or a sheet of a plastic material.

In particular, inner and outer packaging may be made of cardboard or may comprise a plastic material featuring numerous folded portions to form a packaging assembly of box-like shape. When integrating the inner packaging into the outer packaging, e.g. by respectively folding a cardboard or plastic blank, a rather cost- and resource sensitive way of manufacturing can be provided.

In another embodiment, the inner packaging is positively engageable with the interior of the outer packaging by means of the inner packaging's fixing flap portions. Typically, the inner volume of the outer packaging is adapted and configured in accordance with the outer dimensions of the inner packaging in such a way, that the inner packaging can be completely fitted into the outer packaging in such a way, that the fixing flap portions of the inner packaging, in particular of its bottom portion, are folded, e.g. onto or towards oppositely located inside facing front faces of the outer packaging.

Moreover, the inside facing portion of the outer packaging's front faces typically comprise a somewhat uneven or recessed structure so as to positively engage with the free end of the inner packaging's fixing flap portions. Typically, the fixing flap portions are folded upwardly, towards the lid portion upon insertion of the inner packaging into the outer packaging. Upon reaching a mutual abutment configuration of the inner packaging's bottom portion with the outer packaging's bottom portion, the upwardly bended fixing flap portions of, e.g. the inner packaging's bottom portion may relax and bend outwardly and may thus engage with e.g. a lower edge of at least one flap portion extending along the inside portion of the outer packaging's front face. This engagement may further be accompanied and may coincide with an audible click-like noise indicating to a user, that inner and outer packaging mutually engaged.

According to a further embodiment, the outer packaging comprises a somewhat similar overall geometry compared to the inner packaging except that the outer packaging's lateral dimensions are substantially larger than respective lateral dimensions of the inner packaging since the outer packaging is adapted to receive both, the inner packaging as well as a second item, such like a second drug delivery device.

Consequently, the outer packaging also comprises a bottom portion, a first sidewall portion and a second sidewall portion, wherein said sidewall portions extend along opposite sides of the bottom portion. Moreover, the outer packaging's first sidewall portion comprises at least one foldable flap portion overlapping with a foldable flap portion of the outer packaging's second sidewall portion. Alternatively, said foldable flap portion of the outer packaging's first sidewall portion may also directly overlap and engage with the second sidewall portion to form a front face of the outer packaging.

Additionally, the at least one inner packaging's foldable fixing flap portion abuts and engages with the interior of said front face of the outer packaging and/or with the at least one foldable flap portion of at least one outer packaging's sidewall portion.

Typically, the at least one foldable flap portion provided at a longitudinal end of at least one of first and second outer packaging's sidewall portions is located at a predefined distance from a longitudinal end of the outer packaging's bottom portion. In this way, the folded flap portions of at least one of the outer packaging's first and second sidewall portions form an inwardly extending protrusion or protruding structure on the inside facing portion of the outer packaging's front face. The size or the thickness of this inward facing protrusion typically corresponds to the thickness of the sheet material the packaging, and hence first and second sidewall portions are made of.

When first and second sidewall portions both comprise foldable flap portions overlapping and forming the front face, the size or thickness of these inwardly facing protrusions may be about twice the thickness of the sheet material, the outer packaging is made of. The at least one foldable fixing flap portion of the inner packaging's comprises such a geometry, that it snugly fits into the recessed structure formed between the outer packaging's bottom portion and the inside facing protrusion provided and formed by overlapping flap portions of first and/or second sidewall portions of the outer packaging.

Typically, the inner packaging comprises two fixing flap portions located on opposite ends of the inner packaging's bottom portion. In this way, the inner packaging can be inserted into the outer packaging by folding both fixing flap portions upwardly to engage with respective recessed portions of the inside wall structure of the outer packaging.

Here, it is of particular benefit when the fixing flap portions feature a pre-defined flexibility or flexural strength. Hence, an upwardly directed folding or bending of the fixing flaps may occur against a restoring force. As soon as the fixing flap coincide with a recessed wall structure of the outer packaging the fixing flap may relax into said recesses and may non-releasably engage with the outer packaging's recessed structure.

In general it is also within the scope of the present invention, that the outer packaging comprises at least one fixing flap portion to positively engage with a recessed structure of the inner packaging when the inner packaging is inserted into the outer packaging. Hence, at least one or both of inner and outer packaging may comprise at least one fixing flap portion. It is even conceivable that fixing flaps of inner and outer packagings mutually engage upon insertion of the inner packaging into the outer packaging.

In a further embodiment, the inner and/or the outer packaging comprises a lid or lid portion to provide access to their interior. When providing separate lids or comparable closure means, access to the first drug delivery device requires to open the lid of the outer packaging in a first step and to open a respective lid of the inner packaging thereafter, in a second step. In contrast to that, access to the second drug delivery device is already given by simply opening of the outer packaging's lid.

When the inner packaging comprises an own lid to gain access to its interior, a separation of inner packaging and outer packaging may become superfluous. In such configurations it may be beneficial, when the inner packaging is non-releasably attached to the outer packaging. In this context it is even conceivable, that the inner packaging and the outer packaging are integrally formed.

According to another embodiment the inner packaging comprises a lid portion extending along the second sidewall portion. Typically, the lid portion is pivotable along a lid axis or pivot axis extending along the upper end of the second sidewall portion. In this way, the lid portion can be pivoted in order to selectively provide access to the interior of the inner packaging. Additionally and according to another embodiment, the lid portion also comprises a lateral flap portion overlapping with the first sidewall.

When overlapping with the first sidewall, the flap portion may either positively and releasably engage with the first sidewall portion. Alternatively, it is also conceivable, that the lid portion's lateral flap portion is adhesively attached to a portion of the first sidewall. The flap portion or the first sidewall portion may further comprise a structurally weakened portion, by way of which an interconnection of the lateral flap portion and the first sidewall portion may be abrogated or destroyed if required, e.g. for gaining access to the inner packaging's interior.

Said flap portion of the lid portion may form a lateral end section of the blank of sheet material, of which the present packaging is made from. An opposite lateral end of the blank may be provided by the first sidewall portion to at least partially overlap with the lid portion's lateral flap portion.

According to another embodiment, the lid portion also comprises at least a first longitudinal flap portion extending from a longitudinal end thereof and overlapping with at least one of the sidewall's flap portions. In this way, also the longitudinal flap portion of the lid portion may contribute to the formation of the longitudinal front face of the packaging. Hence, the front face as already mentioned above may be formed and provided by the lid portion's first longitudinal flap portion overlapping at least partially with first and/or second flap portions of first and/or second sidewall portions of the inner packaging. By providing such a threefold overlapping configuration at the front face, a rather stable and robust front face of the packaging can be provided.

According to another embodiment, the lid portion also comprises at least a first longitudinal extension extending from a longitudinal end of its first longitudinal flap portion. Typically, the lid portion extends substantially perpendicular to the adjacently located first flap portion. In a similar way also the first longitudinal extension extends substantially perpendicular to the first longitudinal flap portion of the inner packaging's lid portion. Since folding lines or pivot axes extending between and separating flap portion, lid portion and longitudinal extension align substantially parallel, the first longitudinal extension of the lid portion extends substantially parallel to the lid portion and hence substantially parallel to the bottom portion.

Typically, the at least first longitudinal extension of the lid portion at least partially abuts and overlaps with the bottom portion. Therefore, the lid portion itself may e.g. adhesively attach to the bottom portion. In this way, overall stability of the inner packaging and in particular of its front face can be further enhanced. By means of a rather rigid and stiffened front face, the at least one inner packaging's foldable fixing flap portion may experience a respective mechanical support in a direction parallel to the surface normal of the front face. In this way, the positive engagement of e.g. the upwardly bended foldable fixing flap portions with the interior or inside facing front face structure of the outer packaging can be stabilized and structurally reinforced.

Moreover, and according to another embodiment the lid portion comprises first and second longitudinal flap portions at opposite longitudinal ends thereof. In this way, a rather symmetric configuration of the inner packaging can be attained. Additionally, first and second longitudinal flap portions may both comprise first and second longitudinal extensions allowing to fix and to attach both longitudinal end sections of the lid portion directly to the respective longitudinal ends of the inner packaging's bottom portion.

Alternatively it is also conceivable that the at least first extension of the lid portions does not extend inwardly to engage with the bottom portion but does extend outwardly from the front face to form or to contribute to the at least one fixing flap portion.

According to another embodiment the lid portion also comprises a foldable or removable strip portion to provide access to the interior of the inner packaging. Since the lid portion may be directly interconnected with the bottom portion, lifting of the lid portion, e.g. by raising the lateral flap portion of the lid portion into an elevated position with regard to the bottom portion may not be attainable. Instead, by providing a foldable or removable strip portion in or on the lid portion, the lid portion itself may provide a frame structure that remains attached to the sidewall portions and/or to the bottom portion of the inner packaging while the foldable or removable strip portion may be folded or torn away for establishing a through opening in the lid portion that allows to remove the item provided in the inner packaging.

In a further embodiment, said strip portion may also comprise a gripping flap which at least partially protrudes from one of the first and second sidewall portions. Having a gripping flap integrally formed with the foldable or at least partially removable strip portion and having the gripping flap at least partially extending from e.g. an upper edge or upper end of first and/or second sidewall portions, the gripping flap and hence the strip portion integrally formed therewith can be easily gripped in order to raise or to fold the strip portion for gaining access to the inner packaging's interior.

According to another embodiment, the interior of the outer packaging and/or the interior of the inner packaging is divided into at least two compartments by means of at least one partition wall. The partition wall may be arranged in such a way, that even access to the inner packaging is completely blocked as long as the second drug delivery device is located in the outer packaging. In a further embodiment, the inner packaging may comprise a floor space being almost identical to the floor space of the outer packaging.

Here, the inner packaging may be tightly arranged in the outer packaging. Then, the at least one partition wall may be assembled on top of the inner packaging thereby forming a receptacle to receive the second drug delivery device, e.g. on top of the partition wall. Finally, the outer packaging may be closed by a lid located opposite to the floor space or opposite to the bottom of the outer packaging.

In this embodiment, the partition wall may be removably arranged in the outer packaging. When opening the outer packaging only the second drug delivery device is accessible. After a removal of the second drug delivery device the partition wall may be either destroyed or taken out of the outer packaging, e.g. by means of at least one flap attached to the partition wall. After removal of the at least one partition wall, access to the interior packaging and hence access to the first drug delivery device can be provided.

Apart from an arrangement of first and second drug delivery devices on top of each other it is also conceivable to arrange the second drug delivery device next to the inner packaging containing the first drug delivery device. When opening the outer packaging, the user or patient may be inherently informed that the outer packaging serves to accommodate at least two, namely first and second drug delivery devices.

According to another embodiment, the partition wall is operable or designed to impede access to the inner packaging and/or access to the first drug delivery device. When the inner packaging comprises a partition wall said partition wall may correspondingly impede access to an inner packaging's compartment. In a particular embodiment, it is even conceivable, that the partition wall together with adjacent sidewall or bottom wall portions of the outer packaging forms the inner packaging. Consequently, inner packaging and outer packaging may comprise at least one common sidewall portion. Hence, at least a portion of inner packaging and outer packaging may be integrally formed and may be provided by a single sidewall, bottom wall or top wall structure.

It is even within the scope of the present invention that the inner packaging is formed by at least one partition wall extending between two oppositely located side wall portions of the outer packaging.

According to another embodiment, an inside-facing wall portion of the outer packaging is provided with a label. The label may comprise and provide operation instructions of the first and/or the second drug delivery device as well as instructions regarding the sequential use of first and second drug delivery devices. Additionally, the label may be indicative about the order, in which the first and second drug delivery devices are to be used. Moreover, the label may be indicative of an opening procedure for opening the inner packaging.

In still another embodiment, also an outside-facing wall portion of the inner packaging may be provided with a label. This label attached to the inner packaging may be indicative to the user, that it is the second drug delivery device which is to be used prior to opening the inner packaging and hence prior to make use of the first drug delivery device located therein.

Additionally, inner and outer packagings may also be provided with visually perceptible indicators by way of which the order of use of first and second drug delivery devices can be intuitively communicated to a user. Hence, the second drug delivery device and the inner packaging may distinguish by their colour and/or by surface finish. Moreover, the outer circumference of the second drug delivery device may comprise a roughness distinguishing from the roughness of the inner packaging.

In this way, the inner packaging and the second drug delivery device may also be distinguishable in a tactile way. Consequently, even persons suffering impaired vision may unequivocally distinguish the inner packaging and the second drug delivery device.

According to another embodiment, the first drug delivery device is actually arranged in the inner packaging and the second drug delivery device is actually arranged in the outer packaging. Even though the packaging assembly is just designed to receive first and second drug delivery devices in inner and outer packagings, respectively, the packaging assembly may be readily equipped with respective drug delivery devices when commercially distributed to medical staff or patients. While the inner packaging accommodates the first drug delivery device the outer packaging accommodates the inner packaging and the second drug delivery device. Here, the second drug delivery device is arranged inside the outer packaging but outside the inner packaging.

In order to prevent a damage of first and/or second drug delivery devices, at least one or even both drug delivery devices may be detachably fixed in the inner packaging and in the outer packaging, respectively. Here, the inner packaging as well as the outer packaging may comprise and feature respective fixing members, such like clamps, clips or fixing straps.

In particular, the packaging assembly features a nested arrangement of an inner and an outer packaging. When opening the outer packaging, only the second drug delivery device is directly accessible to be used while the first drug delivery device remains packed and sealed by the inner packaging contained in the outer packaging. Such an interleaved configuration of inner and outer packaging provides an intuitive approach to indicate to a user, that the second drug delivery device should be used prior to the first drug delivery device, which is separately packed by the inner packaging.

When opening the outer packaging there is only one, namely the second drug delivery device of a plurality of drug delivery devices, which is ready to use. The user may then intuitively take the second drug delivery device. Hence, a risk of interchanging first and second drug delivery devices can be reduced, simply by wrapping the first drug delivery device in a separate inner packaging.

Naturally, the packaging assembly with its inner packaging and outer packaging is by no way limited to accommodate only two, namely a first and a second drug delivery device. The packaging assembly is particularly adapted and extendable to accommodate and to receive a plurality of drug delivery devices, e.g. three, four, five or even more drug delivery devices.

According to another embodiment at least a third drug delivery device is arranged in the inner packaging or in the outer packaging. For instance, the inner packaging accommodates the first and the third drug delivery device while the outer packaging accommodates the inner packaging and the second drug delivery device. Alternatively, the inner packaging only accommodates the first drug delivery device while the outer packaging accommodates the inner packaging and second and third drug delivery devices located outside the inner packaging. Irrespective of how first, second and third drug delivery devices are distributed and arranged inside the inner and outer packaging first, second and third drug delivery devices are differently packed or wrapped inside the outer packaging and/or inside or by way of the inner packaging.

The differently packed or wrapped first, second and third drug delivery devices inherently indicate to a user or patient the sequence according to which first, second and third drug delivery devices should be used. In particular, the second drug delivery device may be assembled and arranged inside the outer packaging in a rather unwrapped or unpacked way. It may be arranged inside the outer packaging next to or above the inner packaging in a way in which it is ready to use or almost ready to use.

Here, it is conceivable, that the second drug delivery device is provided with a tamper evident closure, which has to be removed prior to use the respective device. Typically, first, second and third drug delivery devices are packed or wrapped in such a way, that removal of the second drug delivery device from the outer packaging provides access to the first and/or third drug delivery device. Also here, first and third drug delivery devices may be differently packed or wrapped inside or outside the inner packaging but always inside the outer packaging. It is conceivable that for instance removal of the third drug delivery device is required and necessary in order to obtain access to the first drug delivery device which is located inside the outer or inner packaging.

In typical embodiments a whole series of drug delivery devices may be packed or wrapped inside the outer packaging and/or inside the inner packaging in a convoluted or nested way. Typically, access to the first drug delivery device requires removal of at least the second and/or the third drug delivery device. It is particularly conceivable that besides the second drug delivery device all residual drug delivery devices are individually packed in an inner packaging, wherein the series of inner packagings is mutually engaged in such a way, that opening of e.g. a first inner packaging requires opening and/or removal of a second inner packaging.

In addition, the geometric size of various inner packagings or compartments thereof may vary in order to visually indicate to a user a predefined sequence of use of the various drug delivery devices. For instance, various lid portions, each of which adapted to cover at least a portion of an inner packaging may feature different sizes so as to reveal different portions of respective drug delivery devices that are located in respective inner packagings. The differently sized lids of inner packagings or of compartments thereof may also directly indicate to a user a sequence of use of the various drug delivery devices.

According to another embodiment the first drug delivery device is fastened by means of a first removable lid. At least one of the second or third drug delivery devices is fastened by means of a second removable lid. Here, first and second lids may at least partially overlap. In this way, removing or opening of for instance the first lid requires a prior removal or opening of a second lid. In this way the type of packaging forces and obliges the user to remove the second lid prior to remove the first lid. Removal of the second lid may provide instant access to the second or third drug delivery device. The user may be then inherently encouraged to only make use of the respective drug delivery device. When for instance a content of the drug delivery device has been used up the user may then proceed to remove the first removable lid to gain access to the first drug delivery device, which is to be used last.

The removable first and/or second lids may either belong or connected to the at least one inner packaging, to a series of inner packagings and/or to the outer packaging. If there are several, i.e. two or more inner packagings arranged inside an outer packaging, the inner packagings may be mechanically engaged or coupled by means of at least one of first and second lids. Moreover, at least one of the inner packagings may be mechanically engaged or mechanically coupled to the outer packaging by means of a removable lid, which may be initially connected to both, the outer packaging and to at least one of the inner packagings.

According to another embodiment at least two drug delivery devices are fastened in the outer packaging and in the inner packaging by means of a fastening member. Here, the fastening member extends across the inner packaging and extends at least partially across the interior of the outer packaging. It may even entirely extend across the interior of the outer packaging. In particular, the fastening member may extend between mutually opposing sidewall portions of the inner packagings and/or of the outer packaging. In this way, the second drug delivery device, which is located outside the inner packaging, and the inner packaging with a further drug delivery device accommodated therein can be fixed and fastened inside the outer packaging by means of a single fastening member, such like a fastening strap.

In such a configuration it is even conceivable, that the inner packaging is substantially lidless while the fastening member effectively acts as and/or provides a lid at least partially extending across the inner packaging. Typically, the fastening member comprises a fastening strap or the like extending between oppositely located sidewall portions of the at least one inner packaging and/or of the outer packaging. In portions overlapping with the second drug delivery device, with a compartment of inner or outer packaging or in portions overlapping with an inner packaging the fastening member may feature different visual appearances and/or different geometric shapes. In these regions the fastening member may exhibit different geometries, in particular different sizes in order to visually indicate to a user a predefined sequence of using the at least first and second drug delivery devices.

Accordingly and following a further embodiment, first and second lids that are designed to at least partially cover at least one of the first, second or third drug delivery devices are different in size. The size of the removable lids may inherently indicate to a user a predefined sequence of use of the drug delivery devices that are at least partially covered by first and second lids, respectively. Moreover, when the lids or the fastening portions are provided with lines of weakening or scoring lines, differently sized lids or different fastening portions may exhibit and feature differently sized or differently behaving scoring lines or respective weakened portions. In this way different lids and/or fastening portions can be equipped and furnished with different mechanical resistance against opening or tear away.

In still another embodiment, the first and/or the second drug delivery device comprise an injection device containing an injectable medicament, such like a pen-type injector comprising a drive mechanism and a cartridge containing the medicament. Such a pen-injector which is operable to repeatedly set and to dispense a dose of predefined or variable size may also be of disposable type and may be intended to be discarded after a single use or after repeated usage. First and/or second drug delivery devices may also comprise a pre-filled syringe adapted for single or multiple use.

In other embodiments at least one of first and second drug delivery devices is designed and comprises a reusable pen-type injector, wherein a cartridge filled with the medicament is removably and interchangeably arranged in the respective device.

At least one of the first and second drug delivery devices is or can be at least partially filled with a medicament to be dispensed by the respective drug delivery device. In particular, the drug delivery device may comprise a prefilled syringe having a medicament or a respective pharmaceutical active substance provided therein. In an alternative embodiment the drug delivery device may also comprise an inhaler, e.g. pre-filled with a medicament.

In another embodiment, the first and the second drug delivery devices comprise different drug formulations. In a treatment plan where the two different drug formulations are needed in a subsequent order, the invention helps to prevent mix-ups and can help to improve as well patient compliance and treatment safety.

In one further embodiment, the drug formulations have at least one common active ingredient. One example is that of two different concentrations of the same active ingredient(s). For some treatments it is appropriate to ramp up the concentration of the active ingredient(s) over time. The invention helps to clearly separate the drug delivery device with the lower concentration from the one with the higher concentration.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:
H-(Lys)4-des Pro36, des Pro37 Exendin-4(1-39)-NH2,
H-(Lys)5-des Pro36, des Pro37 Exendin-4(1-39)-NH2,
des Pro36 Exendin-4(1-39),
des Pro38 [Asp28] Exendin-4(1-39),
des Pro38 [IsoAsp28] Exendin-4(1-39),
des Pro38 [Met(O)14, Asp28] Exendin-4(1-39),
des Pro38 [Met(O)14, IsoAsp28] Exendin-4(1-39),
des Pro38 [Trp(O2)25, Asp28] Exendin-4(1-39),
des Pro38 [Trp(O2)25, IsoAsp28] Exendin-4(1-39),
des Pro38 [Met(O)14 Trp(O2)25, Asp28] Exendin-4(1-39),
des Pro38 [Met(O)14 Trp(O2)25, IsoAsp28] Exendin-4(1-39); or
des Pro38 [Asp28] Exendin-4(1-39),
des Pro38 [IsoAsp28] Exendin-4(1-39),
des Pro38 [Met(O)14, Asp28] Exendin-4(1-39),
des Pro38 [Met(O)14, IsoAsp28] Exendin-4(1-39),
des Pro38 [Trp(O2)25, Asp28] Exendin-4(1-39),
des Pro38 [Trp(O2)25, IsoAsp28] Exendin-4(1-39),
des Pro38 [Met(O)14 Trp(O2)25, Asp28] Exendin-4(1-39),
des Pro38 [Met(O)14 Trp(O2)25, IsoAsp28] Exendin-4(1-39),
wherein the group -Lys6-NH2 may be bound to the C-terminus of the Exendin-4 derivative;

or an Exendin-4 derivative of the sequence
des Pro36 Exendin-4(1-39)-Lys6-NH2 (AVE0010),
H-(Lys)6-des Pro38 [Asp28] Exendin-4(1-39)-Lys6-NH2,
des Asp28 Pro36, Pro37, Pro38Exendin-4(1-39)-NH2,
H-(Lys)6-des Pro36, Pro38 [Asp28] Exendin-4(1-39)-NH2,
H-Asn-(Glu)5des Pro36, Pro37, Pro38 [Asp28] Exendin-4(1-39)-NH2,
des Pro36, Pro37, Pro38 [Asp28] Exendin-4(1-39)-(Lys)6-NH2,
H-(Lys)6-des Pro36, Pro37, Pro38 [Asp28] Exendin-4(1-39)-(Lys)6-NH2,
H-Asn-(Glu)5-des Pro36, Pro37, Pro38 [Asp28] Exendin-4(1-39)-(Lys)6-NH2,
H-(Lys)6-des Pro38 [Trp(O2)25, Asp28] Exendin-4(1-39)-Lys6-NH2,
H-des Asp28 Pro36, Pro37, Pro38 [Trp(O2)25] Exendin-4(1-39)-NH2,
H-(Lys)6-des Pro36, Pro37, Pro38 [Trp(O2)25, Asp28] Exendin-4(1-39)-NH2,
H-Asn-(Glu)5-des Pro36, Pro37, Pro38 [Trp(O2)25, Asp28] Exendin-4(1-39)-NH2,
des Pro36, Pro37, Pro38 [Trp(O2)25, Asp28] Exendin-4(1-39)-(Lys)6-NH2,
H-(Lys)6-des Pro36, Pro37, Pro38 [Trp(O2)25, Asp28] Exendin-4(1-39)-(Lys)6-NH2,
H-Asn-(Glu)5-des Pro36, Pro37, Pro38 [Trp(O2)25, Asp28] Exendin-4(1-39)-(Lys)6-NH2,
H-(Lys)6-des Pro38 [Met(O)14, Asp28] Exendin-4(1-39)-Lys6-NH2,
des Met(O)14 Asp28 Pro36, Pro37, Pro38 Exendin-4(1-39)-NH2,
H-(Lys)6-desPro36, Pro37, Pro38 [Met(O)14, Asp28] Exendin-4(1-39)-NH2,
H-Asn-(Glu)5-des Pro36, Pro37, Pro38 [Met(O)14, Asp28] Exendin-4(1-39)-NH2,
des Pro36, Pro37, Pro38 [Met(O)14, Asp28] Exendin-4(1-39)-(Lys)6-NH2,
H-(Lys)6-des Pro36, Pro37, Pro38 [Met(O)14, Asp28] Exendin-4(1-39)-(Lys)6-NH2,
H-Asn-(Glu)5 des Pro36, Pro37, Pro38 [Met(O)14, Asp28] Exendin-4(1-39)-(Lys)6-NH2,
H-Lys6-des Pro38 [Met(O)14, Trp(O2)25, Asp28] Exendin-4(1-39)-Lys6-NH2,
H-des Asp28 Pro36, Pro37, Pro38 [Met(O)14, Trp(O2)25] Exendin-4(1-39)-NH2,
H-(Lys)6-des Pro36, Pro37, Pro38 [Met(O)14, Asp28] Exendin-4(1-39)-NH2,
H-Asn-(Glu)5-des Pro36, Pro37, Pro38 [Met(O)14, Trp(O2)25, Asp28] Exendin-4(1-39)-NH2,
des Pro36, Pro37, Pro38 [Met(O)14, Trp(O2)25, Asp28] Exendin-4(1-39)-(Lys)6-NH2,
H-(Lys)6-des Pro36, Pro37, Pro38 [Met(O)14, Trp(O2)25, Asp28] Exendin-4(S1-39)-(Lys)6-NH2,
H-Asn-(Glu)5-des Pro36, Pro37, Pro38 [Met(O)14, Trp(O2)25, Asp28] Exendin-4(1-39)-(Lys)6-NH2;
or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (−150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the art that various modifications and variations can be made to the present invention without departing from the spirit and scope of the invention. Further, it is to be noted, that any reference signs used in the appended claims are not to be construed as limiting the scope of the present invention.

### BRIEF DESCRIPTION OF THE DRAWINGS

In the following embodiments the invention will be described in greater detail by making reference to the drawings, in which:

FIG. 1 schematically illustrates the packaging assembly with a second drug delivery device and with an inner packaging disposed therein,

FIG. 2 is illustrative of a schematic cross-section through the packaging assembly in an initial and substantially closed configuration,

FIG. 3 shows the packaging assembly after removal of first and second drug delivery devices,

FIG. 4 shows an alternative embodiment, in which first and second drug delivery devices are arranged on top of each other,

FIG. 5 shows a top view of a preconfigured blank of sheet material to be folded to form an inner packaging featuring a bottom portion with two oppositely located foldable fixing flap portions,

FIG. 6 is a schematic and perspective illustration during insertion of the inner packaging into an outer packaging,

FIG. 7 shows an enlarged view of the inner packaging positively engaged and non-removably arranged inside the inner packaging,

FIG. 8 shows another embodiment of an outer packaging accommodating three drug delivery devices,

FIG. 9 shows an alternative embodiment with three drug delivery devices,

FIG. 10 shows a further embodiment of a packaging assembly accommodating three drug delivery devices,

FIG. 11 is illustrative of another embodiment to accommodate five drug delivery devices,

FIG. 12 shows a further embodiment, wherein three drug delivery devices are located next to each other but at different height levels and

FIG. 13 shows a further embodiment with three drug delivery devices arranged in a convoluted or nested way on top of each other inside an outer packaging.

### DETAILED DESCRIPTION

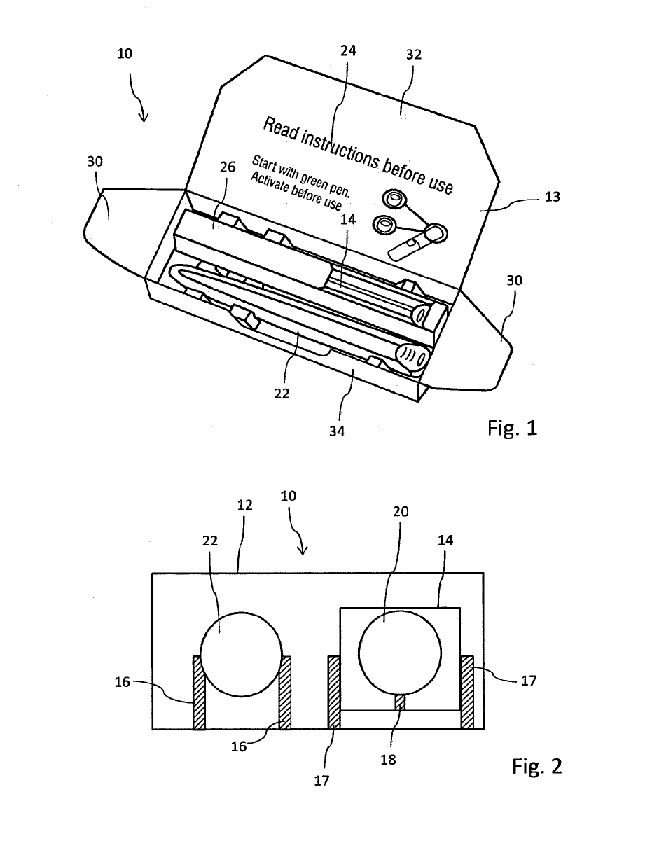

The packaging assembly 10 as illustrated in FIGS. 1-4 comprises an outer packaging 12 featuring a box-like shape. The outer packaging 12 of cubic shape comprises an interior volume, in which an inner packaging 14 and a second drug delivery device 22 is arranged. As shown in cross-section according to FIG. 2, the second drug delivery device 22 is fixed inside the outer packaging 12 by means of respect fastening members 16. Said fastening members 16 may be adapted to frictionally or to positively engage with the outer circumference of the second drug delivery device 22. The fastening members 16 may for instance comprise one or several clips.

In this way, the second drug delivery device 22 may be reversibly removed from the outer packaging 12, e.g. during an initialising treatment with a medicament provided in the respective second drug delivery device 22.

The inner packaging 14 may also be removably fastened inside the outer packaging 12 by means of separate fastening members 17, e.g. provided on diametrically opposite sides of the inner packaging 14. The inner packaging 14 is designed to accommodate a first drug delivery device 20 which is also provided with a respective medicament different from the medicament provided and contained in the second drug delivery device 22. Typically, the medicament contained in the first drug delivery device is to be used and to be administered after a treatment with the medicament provided in the second drug delivery device 22 has terminated.

The inner packaging 14 therefore serves to completely enclose the first drug delivery device 20. When opening the outer packaging 12 initially, e.g. by lifting of a respective lid 13 the user may be confronted with a situation as illustrated in FIG. 1. Here, the second drug delivery device 22, which is to be used initially, is ready to use and can be taken out of the outer packaging 12. It is only after e.g. repeated use of the second drug delivery device 22, that the inner packaging 14 should be opened in order to provide access to the first drug delivery device 20.

The outer packaging 12 as illustrated for instance in FIG. 1 may comprise a cardboard box. It may feature rectangular-shaped sidewalls and foldable flaps 30 at opposite longitudinal ends. The lid 13 may further be provided with a tongue 32 at its free end to engage with an oppositely located sidewall 34 for repeatedly opening and closing the outer packaging 12.

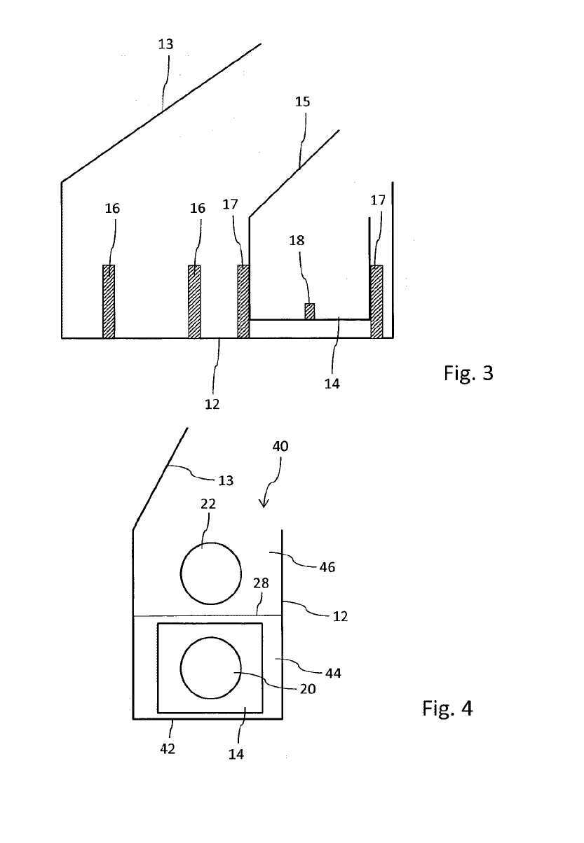

As schematically illustrated in FIG. 3, also the inner packaging 14 may be provided with a lid 15, by way of which the interior volume of the respective inner packaging 14 may become accessible. Moreover, also the inner packaging 15 may comprise a fastening member 18, e.g. at a bottom portion thereof, to engage and to fix the first drug delivery device 20 inside.

In configurations or embodiments, wherein the inner packaging 14 is also provided with a lid 15, it is conceivable that the inner packaging 14 is integrally formed with the outer packaging 12 or that the inner packaging 14 is non-removably connected to the outer packaging 12. In other embodiments it is conceivable, that the inner packaging 14 is substantially lidless and comprises a predetermined breaking structure or respective lines of weakening, by way of which the inner packaging 14 may either be disintegrated or destroyed to gain access to the first drug delivery device 20 located therein. In such configurations it may be beneficial, when the inner packaging 14 is removably attached to the outer packaging 12.

Both packagings, inner packaging 14 and outer packaging 12 may be made of the same packaging material, e.g. cardboard. However, it is also conceivable, that the materials as well as the type of inner and outer packagings 14, 12 vary. The outer packaging 12 comprises a self-supporting box-like shape. In contrast to that, the inner packaging 14 may feature a flexible or elastically deformable flap, envelope or cover by way of which operation of the first drug delivery device can be effectively prevented.

Since the inner packaging 14 with the first drug delivery device 20 located therein is entirely arranged inside the outer packaging 12, the inner packaging 14 does not necessarily have to provide a mechanically protective function. It may therefore comprise or consist of an overwrap or cover.

In FIG. 4, an alternative embodiment of a packaging assembly 40 is illustrated in cross-section. Here, a bottom portion 42 of the outer packaging 12 comprises a surface smaller than the sum of respective bottom faces of inner packaging 14 and second drug delivery device 22. In this way, the two drug delivery devices 20, 22 are to be arranged on top of each other.

It is particularly intended, that the inner packaging 14 is assembled near the bottom 42 of the outer packaging 12. Then, on top of the inner pack 14 there is provided a partition wall 28 thereby dividing the interior of the outer packaging 12 into at least two separate compartments44, 46, each of which being adapted to separately accommodate either a drug delivery device 20, 22 and/or an inner packaging 14 comprising the first drug delivery device 20.

As indicated in FIG. 4, the bottom compartment 44 is adapted to receive the inner packaging 14 and hence the first drug delivery device 20 located therein. The upper compartment 46 formed by the sidewall portions of the outer packaging 12 and the partition wall 28 is adapted to accommodate the second drug delivery device 22.

As a consequence and when opening the outer packaging 12, e.g. by lifting the respective lid 13, restricted access to only one, namely to the second drug delivery device 22 is given. Access to the inner packaging 14 and/or to the first drug delivery device 20 requires removal of the partition wall 28, which is only possible after removal of the second drug delivery device 22.

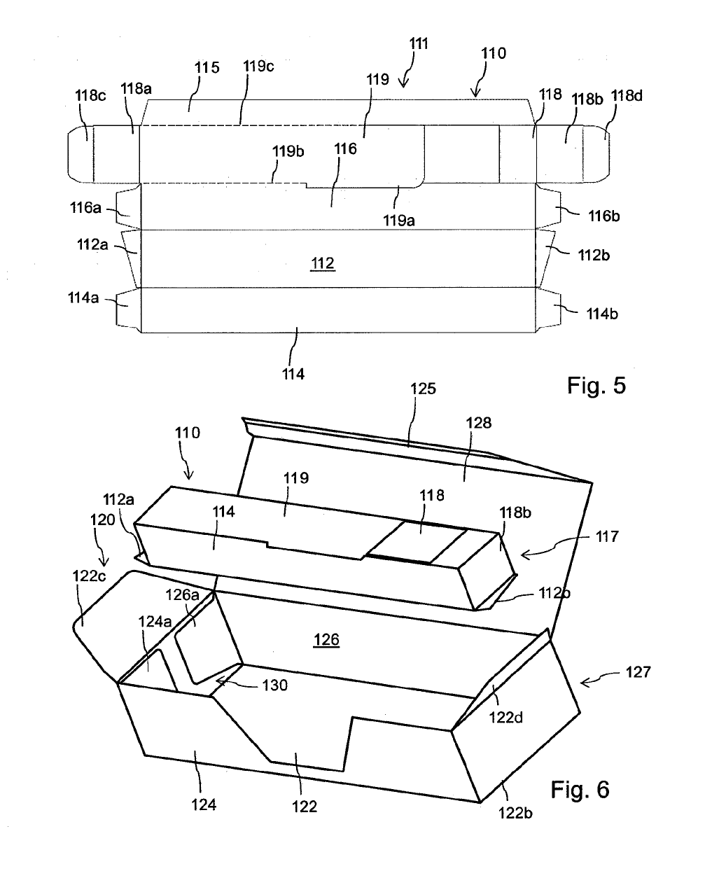

In FIG. 5, a top view of a blank 111 of a sheet material to form an inner packaging 110 is illustrated. The blank 111 and hence the inner packaging 110 comprises a bottom portion 112 of rather rectangular shape which is laterally sandwiched between a first sidewall portion 114 and a second sidewall portion 116. First and second sidewall portions 114, 116 are foldable along the indicated fold lines separating respective sidewall portions 114, 116 from the bottom portion 112.

Adjacent to the second sidewall portion 116, there is provided a lid portion 118 featuring an overall size and geometry that substantially matches the size and geometry of the bottom portion 112. As further indicated in FIG. 5, also first and second sidewall portions 114, 116 comprise substantially identical shape and size, so that a rather rectangular shaped or cubic shaped inner packaging 110 can be provided. The lid portion 118 is further integrally formed with a lateral flap portion 115 forming a lateral end of the blank 111.

When folding first sidewall portion 114, the bottom portion 112, the second sidewall portion 116, the lid portion 118 and the lateral flap portion 115 along the indicated folding lines separating the adjacently located portions 114, 112, 116, 118, 115, the lateral flap portion 115 may at least partially overlap with the first sidewall portion 114. In the this embodiment, said lateral flap portion 115 may be adhesively or releasably attached to the first sidewall portion 114.

As further indicated in FIG. 5, the two sidewall portions 114, 116 each comprise first and second foldable flap portions 114a, 114b, 116a, 116b at their longitudinal ends. Said flap portions 114a, 114b, 116a, 116b extend beyond the lateral end sections of respective sidewall portions 114, 116. As indicated in FIG. 5, the foldable flap portions 114a, 114b, 116a, 116b are integrally formed with respective first and second sidewall portions 114, 116.

Additionally, also the lid portion 118 comprises first and second foldable flap portions 118a, 118b. Moreover, said longitudinal flap portions 118a, 118b may further extend into longitudinal extensions 118c, 118b. While the longitudinal flap portions 118a, 118b of the flap portion may substantially form a front face 117 of the packaging 110, the respective first and second longitudinal extensions 118c, 118b may substantially overlap with an end section of the bottom portion 112. In this way, the lid portion 118 may be directly engaged and fixed to the bottom portion 112.

Additionally, the oppositely located foldable flap portions 116a, 116b of the second sidewall portion 116 as well as oppositely located and longitudinally separated foldable flap portions 114a, 114b of the first sidewall portion may either at least partially and mutually overlap or may additionally overlap with the longitudinal flap portions 118a, 118b of the lid portion 118.

By means of the various longitudinal and foldable flap portions 114a, 114b, 116a, 116b, 118a, 118b of first and second sidewall portions 114, 116 and of the lid portion 118, a rectangular-shaped three-dimensional packaging 110 can be formed, especially by mutually interconnecting overlapping flap portions 114a, 116a, 118a, 114b, 116b, 118b, e.g. by making use of an adhesive.

Since the lid portion 118 provides and substantially constitutes the longitudinally located front faces 117 by means of its downwardly foldable flap portions 118a, 118b, the lid portion 118 is substantially hindered from folding into a lifted or elevated configuration to provide access to the interior of the packaging 110. However, in order to gain access to the packaging's interior, the lid portion 118 is further equipped with a strip or strap portion 119 featuring a gripping flap 119a extending at least partially from the second sidewall portion 116.

Additionally and as indicated by the dashed lines 119b, 119c, the strip portion 119 may be at least partially torn away or torn off from the lid portion 118. Here, dashed lines 119b, 119c may represent predetermined breaking structures or respective lines of weakening providing a well-defined and pre-determined breaking and separating of the strip portion 119 from the lid portion 118.

Furthermore and as becomes apparent from FIG. 6, the inner packaging 110 comprises longitudinally extending foldable fixing flap portions 112a, 112b extending from opposite longitudinal ends of the bottom portion 112 and extending beyond and outwardly from a lower edge of the front face 117. Hence, the fixing flap portions 112, 112b protrude from the front face 117 of the inner packaging 110.

In the illustrated embodiment the fixing flap portions 112a, 112b are integrally formed as longitudinal extensions of the inner packaging's bottom portion 112. In other embodiments the fixing flap portions may be connected and may be integrally formed with the flap portions 114a, 114b, 116a, 116b, 118a, 118b of the first sidewall portion 114, the second sidewall portion 116 and/or of the lid portion 118.

A secondary or outer packaging 120 as illustrated in FIG. 6 comprises a rather conventional structure. The packaging 120 features a bottom portion 122 having foldable flap portions 122a, 122b that are adapted to form and to provide oppositely located longitudinal front faces 127 of the outer packaging 120. Attached to the inside of said flap portions 122a, 122b there are attached inwardly folded first and second flap portions of sidewall portions 124, 126. In FIG. 6, only the first foldable flap portion 124a of the first sidewall and the first foldable flap portion 126a of the second sidewall portion 126 are illustrated.

As further illustrated in FIG. 6, said first flap portions 124a, 126a are adhesively attached to the interior of the upwardly folded flap portion 122a of the bottom portion 122. Additionally, the bottom portion is further provided with oppositely located longitudinal extensions 122c, 122d that extend upwardly or inwardly from an upper end section of the bottom portion's 122 foldable flap portions 122a, 122b. Similar as already described in view of FIG. 5, the outer packaging's 120 second sidewall portion 126 is further connected with a foldable lid portion 128 featuring a lateral flap portion 125 that is adapted to releasably or non-reversibly engage with the first sidewall portion 124.

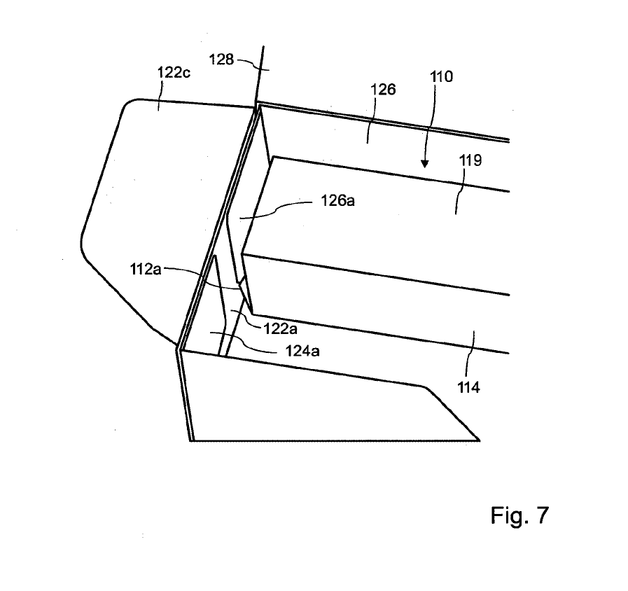

As in particular indicated in the enlarged view according to FIG. 7 the first flap portions 124a, 126a of respective first and second sidewall portions 124, 126 of the outer packaging 120 form a recessed structure at a lower edge thereof towards the longitudinal end of the outer packaging's 120 bottom portion 122. The depth of the recessed structure, which may feature a wedged shape, is governed by the thickness of the sheet material, of which the sidewalls 124, 126 and their foldable flap portions 124a, 126a are made.

The recessed structure 130 provided between a lower edge of the first flap portions 124a, 126a of first and second sidewall portions 124, 126 corresponds to the size and shape of the bottom portion's 112 foldable and longitudinally extending fixing flap portions 112a, 112b of the inner packaging 110. As indicated in FIG. 7, the upwardly folded fixing flap portions 112a, 112b of the inner packaging 110 engage with a lower edge of the first foldable flap portion 126a of the second sidewall 126 of the outer packaging 120.

On the opposite longitudinal end, a corresponding snap-in mechanism is typically provided, so that the inner packaging 110 can be inserted into the outer packaging 20 to attain a positively engaged mutual attachment of the interleaved and nested packagings 110, 120. The upwardly bended foldable fixing flap portions 112a, 112b of the inner packaging's 110 bottom portion 112 engage with the recessed structure 130 in a hooking manner and effectively prevent an uncontrolled release of the inner packaging 110 from the outer packaging 120.

Since longitudinal dimensions of inner packaging 110 and outer packaging 120 mutually match and correspond to each other in such a way, that the inner packaging is snugly fitted inside the outer packaging 120, an uncontrolled or self-acting release of the inner packaging 110 from the outer packaging 120 can be effectively prevented. In effect, the first packaging 110 can be fixedly attached inside the outer packaging without the use of any adhesives. Moreover, once the inner packaging 110 is connected with the outer packaging 120, a destructionless release and removal of the inner packaging 110 from the outer packaging 120 can be effectively prevented and may therefore be excluded.

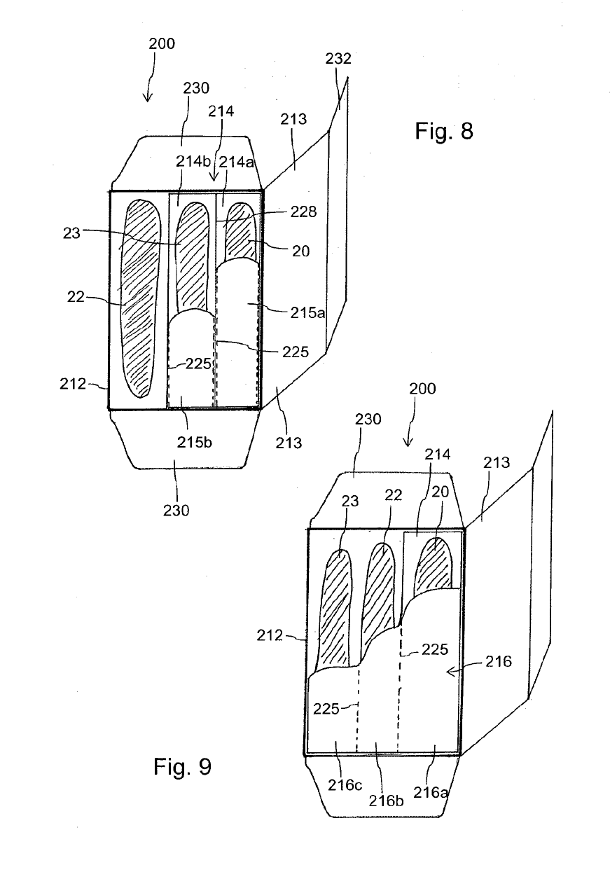

In FIGS. 8, 9 and 10 three further embodiments of a packaging assembly 200 are illustrated featuring an outer packaging 212 of rectangular and box-like shape. The outer packaging 212 comprises a foldable lid 213 to completely cover the interior of the outer packaging 212. Furthermore and as already described in connection with FIG. 1, the outer packaging 212 also comprises foldable flaps 230 that are located on oppositely located sidewalls adjacent to that sidewall which is connected to the foldable lid 213. The lid 213 further comprises a tongue 232 at its free end to engage with an oppositely located sidewall of the outer packaging 212 to enable repeated opening and closing of the outer packaging 212.

Inside the outer packaging 212 as illustrated in FIG. 8 there is accommodated a second drug delivery device 22 and an inner packaging 214. The inner packaging 214 is divided into two compartments 214a, 214b that are separated by partition wall 228. In each one of the compartments 214a, 214b there is accommodated and located a further drug delivery device 20, 23. Here, a first drug delivery device 20 is located in the compartment 214a while a third drug delivery device 23 is located in the second compartment 214b. Each one of the compartments 214a, 214b is provided and at least partially covered with a lid 215a, 215b. The first removable lid 215a covers a portion of the first compartment 214a of the inner packaging 214 while the second removable lid 215b covers only a portion of the second compartment 214b of the inner packaging 214.

As illustrated in FIG. 8, first and second lids 215a, 215b are different in size. As seen from the left to the right, the size of the lids 215b, 215a increases so as to indicate to a user to start with the second drug delivery device 22. Thereafter, the user may tear away the second and comparatively small lid 215b to gain access and to make use of the third drug delivery device 23. Thereafter, the user may then make use of the remaining first drug delivery device 20 after removing the respective residual first lid 215a.

First and second lids 215a, 215b are connected to the sidewalls of the inner packaging 214 and its partition wall 228 by means of predetermined breaking structures 225 or respective lines of weakening. As illustrated in FIG. 8, the second drug delivery device 22, which is to be used first is rather unwrapped or unpacked and is located outside the inner packaging 214 but inside the outer packaging 212.

In the embodiment as shown in FIG. 9, only the first drug delivery device 20 is located in the inner packaging 214 while second and third drug delivery devices 22, 23 are arranged outside the inner packaging 214 but inside the outer packaging 212. In the embodiment according to FIG. 9, the inner packaging 214 is only partially covered by a fastening member 216 extending across a portion of all three drug delivery devices 20, 22, 23. Second and third drug delivery devices 22, 23 as well as the first drug delivery device 20 are arranged substantially parallel with respect to each other.

Moreover, since the drug delivery devices 20, 22, 23 are of almost equal size and longitudinal extension they are arranged next to each other inside the outer packaging 212. The fastening member 216 extends across a lower portion of the third drug delivery device 23, across a lower portion of the second drug delivery device 22 and across a lower portion of the first drug delivery device 20. In respective overlapping regions the fastening member 216 comprises differently sized fastening portions 216c, 216b and 216a. The fastening portion 216a substantially overlaps with the interior of the inner packaging 214 while the fastening portion 216b adjacent to the fastening portion 216a extends across the second drug delivery device 22 while another fastening portion 216c adjacent to the fastening portion 216b extends over a portion of the third drug delivery device 23. As illustrated in FIG. 9, the fastening portion 216b is integrally formed and is located between the fastening portions 216c and 216a.

The fastening portion 216c extending across the third drug delivery device 23 has a smaller extension in longitudinal direction of the drug delivery device 23 compared to the adjacently located fastening portion 216b. In a similar way also the fastening portion 216b is somewhat shorter or smaller than the fastening portion 216a extending across the inner packaging 214. The fastening portions 216c, 216b, 216a may be separable by means of a breaking structure 225, e.g. by means of weakened lines or scoring lines that allow to separate the smallest fastening portion 216c initially, i.e. upon initial use of the packaging assembly 200 and the drug delivery devices 20, 22, 23 located therein.

By means of the breaking structure 225, the smallest fastening portion 216c should be removed first in order to provide access to the third drug delivery device 23. Thereafter, the adjacently located fastening portion 216b may be removed in order to gain access to the second drug delivery device 22. In a last and final step the inner packaging 214 may be taken out of the outer packaging 212 or the fastening portion 216a extending across a portion of the inner packaging 214 may be removed to provide access to the first drug delivery device 20.

The fastening member 216 may comprise a kind of a fastening strap or a foil. The fastening member 216 may be attached or connected to oppositely located sidewall portions, hence, to a left and to a right-handed sidewall portion of the outer packaging 212. In this way and by means of the fastening member 216 the inner packaging 214 may be secured and fastened inside the outer packaging 212. The fastening member 216 may comprise cardboard or a plastic material as well as a plastic foil. It may be connected and attached to the outer packaging 212 in a manifold of different ways. For instance, the fastening member 216 may be adhesively attached or stapled to the outer packaging 212.

In various embodiments the inner packaging 214 may be substantially lidless. Its interior may be at least partially covered by the fastening member 216 which is only attached to the outer packaging 212.

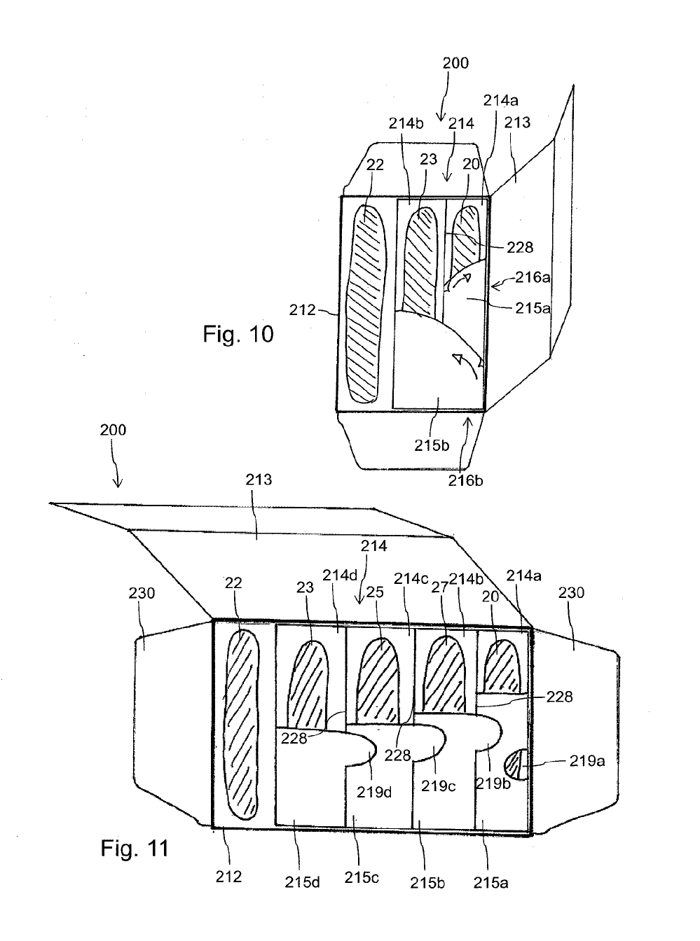

In FIG. 10 another embodiment of a packaging assembly 200 comprising three drug delivery devices 20, 22, 23 is illustrated. There and in comparison to the embodiment as shown in FIG. 8, the inner packaging 214 comprises first and second compartments 214a, 214b, wherein the first compartment 214a accommodates a first drug delivery device 20 and wherein the second compartment 214b accommodates a third drug delivery device 23. The second drug delivery device 22 is directly accommodated in the outer packaging 212 outside the inner packaging 214.

Also here and in comparison to the embodiment as shown in FIG. 8, the first compartment 214a of the inner packaging 214 is partially covered by a removable first lid 215a. A portion of the second compartment 214b is covered by a second removable lid 215b. But as illustrated in FIG. 10 the second lid 215b also at least partially extends across the first lid 215a. Removal of the first lid 215a therefore requires to remove the second lid 215b first. First and second lids 215a, 215b further serve and act as fastening portions 216a, 216b since the lids 215a, 215b serve to keep and to fasten the respective drug delivery devices 20, 23 in their compartments 214a, 214b of the inner packaging 214.

For a user of the packaging assembly 200 and upon opening of the lid 213 the second drug delivery device 22 is the only drug delivery device which is directly accessible. Since the convoluted and overlapping arrangement of first and second lids 215a, 215b is immediately apparent to the user it is inherently clear to the user, that after consumption of the medicament provided by the second drug delivery device 22 it will be the third drug delivery device 23 which is to be used next, since the second lid 215b is the only lid which is directly accessible.

Arranging the second drug delivery device 22 outside the inner packaging and covering first and second compartments 214a, 214b by means of differently sized and mutually overlapping first and second lids 215a, 215b a sequence of intended use of the drug delivery devices 22, 23 and 20 is immediately apparent to the user.

FIG. 11 shows another embodiment, wherein the inner packaging 214 comprises altogether four compartments 214a, 214b, 214c, 214d. Each one of these compartments 214a, 214b, 214c, 214d is partially covered with a respective lid 215a, 215b, 215c, 215d. The compartments 214a, 214b, 214c, 214d together with their respective lids 215a, 215b, 215c, 215d are arranged parallel and next to each other. As shown in FIG. 11 adjacently located lids 215a, 215b, 215c, 215d are different in size so as to indicate the intended sequence of use of the drug delivery devices 22, 23, 25, 27, 20 located in the outer packaging 212 and located in the various compartments 214d, 214c, 214b, 214a of the inner packaging 214. As further illustrated in FIG. 11 three of the lids 215d, 215c, 215b comprise a gripping flap 219d, 219c, 219b extending onto and at least partially across an adjacently located lid 215c, 215b, 215a of the neighboring compartment 214c, 214b, 214a. Also here, and as already described in connection with the embodiment according to FIG. 10, adjacently located lids 215d, 215c, 215b extend at least partially across a neighboring lid 215c, 215b, 215a so as to prevent or to hinder opening of the lids 215a, 215b, 215c, 215d in a wrong sequence.

In the embodiment as shown in FIG. 11 after opening of the outer packaging's 212 lid 213 only the second drug delivery device 22 located outside the inner packaging 214 is directly accessible to a user. After removing and use of the second drug delivery device 22, a user may take the gripping flap 219d of the lid 215d in order to fold away or to remove the lid 215d. Then, access is given to the drug delivery device 23 located in a fourth compartment 214d of the inner packaging 214. Removal of the lid 215d and the drug delivery device 23 then allows to completely remove or to fold open the adjacently located lid 215c by gripping of the gripping flap 219c. Removing or folding open of the lid 215c gives access to the next drug delivery device 25 located in the third compartment 214c of the inner packaging 214.

Thereafter, the lid 215b may be opened by making use of the gripping flap 219b extending onto and over the lid 215 of the first compartment 214a of the inner packaging 214. Removal or folding open of the lid 215b provides access to the drug delivery device 27 before the last, hence the first drug delivery device 20 is to be taken out of the inner packaging 214 by removing or opening of the lid 215a. The lid 215a may comprise a gripping recess 219a or may be provided with a particular gripping flap that distinguishes from gripping flaps 219b, 219c, 219d.

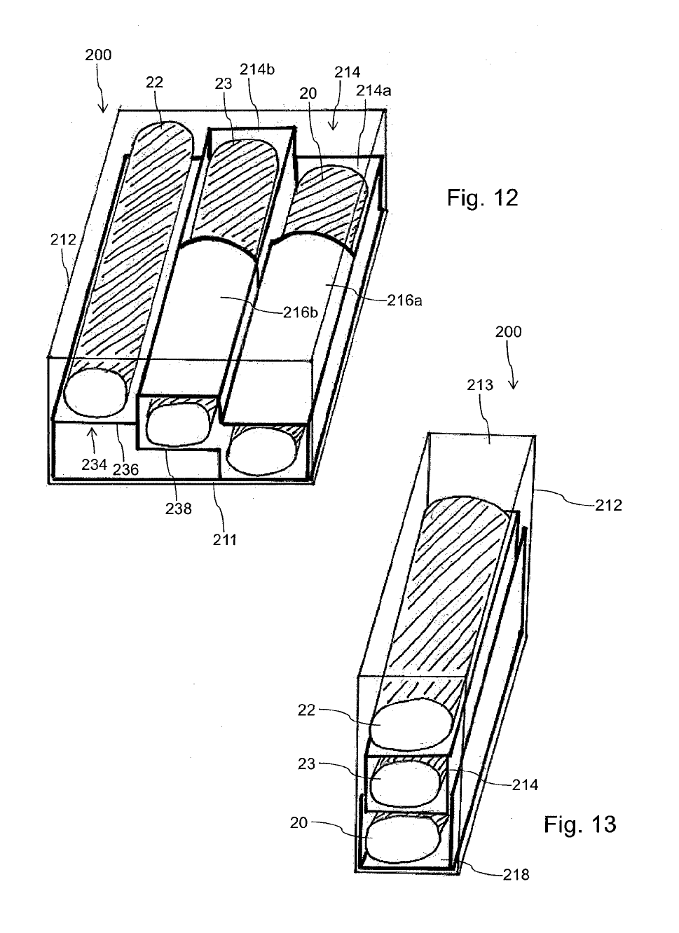

In the embodiment according to FIG. 12 also three drug delivery devices 20, 22, 23 are arranged in a distinguishing way. The outer packaging 212 is illustrated substantially transparent with a closeable lid 213 removed. The outer packaging 212 comprises an insert 234 having a staircase-like profile extending in longitudinal direction of the drug delivery devices 20, 22, 23. The insert 234 forms and provides an upper pedestal portion 236 to support the second drug delivery device 22. Adjacent to the upper pedestal portion 236 the insert 234 comprises a lower pedestal portion 238 to provide support for the third drug delivery device 23. The lower pedestal portion 238 located at a predefined distance from a bottom portion of the outer packaging 212 terminates between the third drug delivery device 23 and the first drug delivery device 20. Hence, the first drug delivery device 20 is located directly on the bottom portion 211 of the outer packaging 212. The inner packaging 214 comprises a shape and structure that accommodates the geometric structure and shape of the insert 234.

The inner packaging 214 comprises a first compartment 214a to accommodate the first drug delivery device 20 and further comprises a second compartment 214b to accommodate the third drug delivery device 23. As can be seen from the cross-section of FIG. 12 first and second compartments 214a, 214b are directly interconnected but they are located at different height levels compared to the bottom portion 211 of the outer packaging 212. The inner packaging 214 may comprise a cardboard structure but may also be made of a flexible or resilient material. As illustrated in FIG. 12 the compartments 214a, 214b of the inner packaging 214 may be open towards the top.

Here, at least a portion of the interior of the compartments 214a, 214b may be covered with fastening portions 216b, 216a of a fastening member 216. As already described in connection with FIG. 9, the fastening portions 216a, 216b, that are typically of different size may be sequentially removed or folded away in order to provide sequential access to the third drug delivery device 23 and to the first drug delivery device 20.

The arrangement of various drug delivery devices 22, 23, 20 at different height levels additionally stipulates and suggests to the user of the packaging assembly 20 to make use of the various drug delivery devices 22, 23, 20 in a well-defined sequential order.

In the embodiment as shown in FIG. 13 two different inner packagings, namely a lower packaging 218 and an upper packaging 214, are arranged on top of each other. Inside the lower inner packaging 218 there is accommodated the first drug delivery device 20, which is to be used last. The lower inner packaging 218 is arranged on the bottom portion 211 of the outer packaging 212. The lower inner packaging 218 is somewhat wider or larger than the upper inner packaging 214 so that the second inner packaging 214 can be arranged on top of the first drug delivery device 20 in a way that the upper inner packaging 214 is partially received in the inner packaging 218. The upper inner packaging 214 is completely closed and features a lid as for instance described in connection with FIG. 3 or 5-7.

The upper inner packaging 214 accommodates a third drug delivery device 23 while a second drug delivery device 22, which is to be used first is located on top of the inner packaging's 214 lid. When opening the upper lid 213 of the outer packaging 212 only the second drug delivery device 22 is directly accessible. Access to the third drug delivery device 23 requires removal of the second drug delivery device 22 and opening of the lid of the upper inner packaging 214. After removing the third drug delivery device 23 and after removal of the inner packaging 214 access is provided to the first drug delivery device 20, which is to be used last.

In an alternative it is also conceivable that the first drug delivery device 20 is arranged outside the inner packaging 218 so that the entire packaging assembly 200 only comprises a single inner packaging 214 which is directly positioned on top of the first drug delivery device 20.

The various embodiments as illustrated and described herein show different ways on how to indicate to a user a sequence of intended use of a series, i.e. of at least two drug delivery devices simply by wrapping or packing the devices in different and distinguishable ways inside an outer packaging.

1-25. (canceled)

26. A packaging assembly to accommodate at least two drug delivery devices and comprising:
an inner packaging to accommodate a first drug delivery device, and
an outer packaging to accommodate a second drug delivery device and to accommodate the inner packaging.

27. The packaging assembly according to claim 26, wherein the inner packaging is adapted to completely enclose the first drug delivery device.

28. The packaging assembly according to claim 26, wherein the outer packaging comprises at least one fastening member to fix the second drug delivery device therein.

29. The packaging assembly according to claim 26, wherein the outer packaging comprises at least one fastening member to fix the inner packaging therein.

30. The packaging assembly according to claim 26, wherein the inner packaging comprises at least one fastening member to fix the first drug delivery device therein.

31. The packaging assembly according to claim 26, wherein the inner packaging comprises:
a bottom portion,
a first sidewall portion and a second sidewall portion extending along opposite sides of the bottom portion,
at least one front face extending between an end of first sidewall portion, second side wall portion and bottom portion, and
at least one fixing flap portion extending as an extension of the bottom portion beyond the front face to engage with an inside of an outer packaging adapted to receive the inner packaging.

32. The packaging assembly according to claim 31, comprising a first and a second fixing flap portion extending from opposite ends of the bottom portion and/or extending from oppositely located front faces.

33. The packaging assembly according to claim 31, wherein the at least one fixing flap portion is integrally formed with the bottom portion.

34. The packaging assembly according to claim 31, wherein at least one of first and second sidewall portions comprises first and/or second flap portions extending from opposite ends of the respective sidewall portions.

35. The packaging assembly according to claim 26, wherein the inner packaging is positively engageable with the interior of the outer packaging by means of the at least one inner packaging's fixing flap portion.

36. The packaging assembly according to claim 31, wherein
the outer packaging comprises a bottom portion, a first sidewall portion and a second sidewall portion extending along opposite sides of the bottom portion, and
wherein the outer packaging's first sidewall portion comprises at least one foldable flap portion overlapping with a foldable flap portion of the outer packaging's second sidewall portion or overlapping with the second sidewall portion to form a front face,
wherein the at least one inner packaging's fixing flap portion abuts and engages with the interior of the front face and/or with the at least one foldable flap portion of at least one outer packaging's sidewall portion.

37. The packaging assembly according to claim 26, wherein outer and/or inner packaging comprise a lid to provide access to the interior of the outer and/or inner packaging respectively.

38. The packaging assembly according to claim 26, wherein the interior of the outer packaging or inner packaging is divided into at least two compartments by means of at least one partition wall.

39. The packaging assembly according to claim 38, wherein the partition wall is operable to impede access to the inner packaging or to impede access to an inner packaging's compartment.

40. The packaging assembly according to claim 26, wherein an inside facing wall portion of the outer packaging is provided with a label.

41. The packaging assembly according to claim 26, wherein an outside facing wall portion of the inner packaging is provided with a label.

42. The packaging assembly according to claim 26, wherein the first drug delivery device is arranged in the inner packaging and wherein the second drug delivery device is arranged in the outer packaging.

43. The packaging assembly according claim 42, wherein at least a third drug delivery device is arranged in the inner packaging or in the outer packaging, and wherein first, second and third drug delivery devices are differently packed or wrapped.

44. The packaging assembly according to claim 42, wherein the first drug delivery device is fastened by means of a first removable lid and wherein at least one of second or third drug delivery devices is fastened by means of a second removable lid and wherein first and second lids at least partially overlap.

45. The packaging assembly according to claim 42, wherein claims at least two drug delivery devices are fastened in the outer packaging and in the inner packaging by means of a fastening member extending across the inner packaging and extending at least partially across the interior of the outer packaging.

46. The packaging assembly according to claim 44, wherein first and second lids are different in size or wherein adjacently located and separable first and second portions of the fastening member are different in size.

47. The packaging assembly according to claim 26, wherein the first and/or the second drug delivery device comprise an injection device.

48. The packaging assembly according to claim 47, wherein at least one of the first and second drug delivery devices contains an injectable medicament.

49. The packaging assembly according to claim 47, wherein the first and the second drug delivery devices comprise different drug formulations.

50. The packaging assembly according to claim 49, wherein the drug formulations have at least one common active ingredient.
